SEQUENCE LISTING (1 sequence)

SEQ ID NO: 1 - protein, 770 aa, Artificial Sequence
  Synthetic
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

(19) United States
(12) Patent Application Publication
(10) Pub. No.: US 2024/0065237 A1
(43) Pub. Date: Feb. 29, 2024

(54) A GENETICALLY MODIFIED IMMUNODEFICIENT MOUSE EXPRESSING HUMAN OR HUMANIZED APP AND MUTATED HUMAN PSEN1

(71) Applicant: The Jackson Laboratory, Bar Harbor, ME (US)

(72) Inventors: Gareth Howell, Bar Harbor, ME (US); Kristen Onos, Bar Harbor, ME (US)

(73) Assignee: The Jackson Laboratory, Bar Harbor, ME (US)

(21) Appl. No.: 18/267,389

(22) PCT Filed: Dec. 14, 2021
(86) PCT No.: PCT/US2021/063306

§ 371 Date: Jun. 14, 2023

Related U.S. Application Data
(60) Provisional application No. 63/126,457, filed on Dec. 16, 2020.

(51) Int. Cl. A01K 67/027; C07K 14/47

(52) U.S. Cl. CPC A01K 67/0275; C07K 14/4711; A01K 2207/15; A01K 2217/054; A01K 2227/105; A01K 2267/0312

(57) ABSTRACT

The present disclosure provides immunodeficient mouse models that comprise a nucleic acid encoding a human or humanized amyloid precursor protein (APP) and, in some models, further comprise a nucleic acid encoding a mutated human presenilin 1 protein (PSEN1). These mouse models are useful, for example, for Alzheimer's disease studies.

### RELATED APPLICATION

This application claims the benefit under 35 U.S.C. § 119(e) of U.S. provisional application No. 63/126,457, filed Dec. 16, 2020, which is incorporated by reference herein in its entirety.

### BACKGROUND

Transgenic mouse models expressing human amyloid precursor protein (APP) with or without the expression of human presenilin 1 (PSEN1) have been used extensively to study Alzheimer's disease (AD) in vivo to gain a better understanding of pathogenesis of the disease in human patients. Nevertheless, such models often inadequately recapitulate the widespread neurodegeneration and regional brain atrophy that occurs in AD (Drummond et al., Acta Neuropathol. 2017 February; 133(2):155-175). Additionally, such models exhibit dramatic differences in neuroinflammation across backgrounds. For example, the microglia response in one mouse model is blunted and the mice lack disease associated microglia, while another mouse model exhibits a robust microglia response. Still other transgenic immunodeficient mouse models expressing APP are inadequate for AD studies because they develop a large tumor burden and cannot be aged beyond eight months (Espuny-Camacho et al., Neuron 2017; 93(5):1066-81).

### SUMMARY

The present disclosure provides, in some aspects, improved immunodeficient mouse models of Alzheimer's disease (AD). In some embodiments, an immunodeficient mouse model of AD expresses a human or humanized amyloid precursor protein (APP). In other embodiments, an immunodeficient mouse model of AD also expresses a mutated human presenilin 1 protein (PSEN1, also abbreviated as PSEN1). Unlike other mouse models of AD, the models provided herein do not develop a large tumor burden (often associated with death at ˜7-8 months), thus they can be aged to a more relevant time point for studying certain mechanisms of development and progression of AD. Modeling A D on an immunodeficient background permits a platform for studying immune interactions with amyloid, offering insight to how reduced immunity impacts short-term memory and/or impacts the development of hippocampal and cortical plaque deposits, for example.

The mouse models provided herein are based, at least in part, on the theory that adaptive immunity has a role in the pathogenesis of AD by modulating neuroinflammation in the brain in response to amyloid. This theory was tested by disrupting adaptive immunity using a two-step approach. A non-obese diabetic (NOD) mouse expressing humanized APP and a mutated PSEN1 was first generated (the “NOD.APP/PSEN1” model). The NOD.APP/PSEN1 model was then crossed to the NOD.Cg-Prkdcs^{scid} Il2rg^{tm1Wjl}/SzJ (NSG™) mouse model to generate a novel immunodeficient mouse model expressing a humanized APP and a mutated human PSEN1 (the “NSG.APP/PSEN1” model).

Thus, some aspects of the present disclosure provide an immunocompromised mouse comprising in its genome a loss-of-function mutation in a murine Prkdc gene, a loss-of-function mutation in a murine Il2rg gene, and a nucleic acid encoding a human or humanized amyloid precursor protein (APP).

In some embodiments, the mouse has a non-obese diabetic (NOD) genetic background. In some embodiments, the loss-of-function mutation in a murine Prkdc gene is a null mutation. For example, the null mutation may be a Prkdc^{scid} mutation. In some embodiments, the loss-of-function mutation in a murine Il2rg gene is a null mutation. For example, the null mutation may be an Il2rg^{tm1Wjl} mutation. In some embodiments, the mouse has a NOD.Cg-Prkdc^{scid} Il2rg^{tm1Wjl}/SzJ genetic background. As another example, the null mutation may be an Il2rg^{em26Cd22} mutation. In some embodiments, the mouse has a NOD-Prkdc^{em26Cd52}Il2rg^{em26Cd22}/NjuCrl genetic background.

In some embodiments, the mouse comprises a nucleic acid encoding a humanized APP. For example, the nucleic acid encoding a humanized APP may be a chimeric nucleic acid comprising mouse and human coding sequences. In some embodiments, the chimeric nucleic acid comprises a human coding sequence in the A-beta domain of a mouse APP coding sequence. In some embodiments, the chimeric nucleic acid encodes human mutations K595N and M596L, relative to a human APP comprising the amino acid sequence of SEQ ID NO: 1. In some embodiments, the mouse comprises in its genome an APPswe transgene.

In some embodiments, the mouse further comprises in its genome a nucleic acid encoding a mutated human presenilin 1 protein (PSEN1). The nucleic encoding a mutated PSEN1 may comprise, for example, a human PSEN1 coding sequence that comprises a deletion in exon 9. In some embodiments, the mouse comprises in its genome an a PSEN1de9 transgene. In some embodiments, the mouse comprises in its genome Tg(APPswe,PSEN1de9)85Dbo transgene insertion.

In some embodiments, the mouse has a characteristic of early-onset Alzheimer's disease. For example, the characteristic of early-onset Alzheimer's disease may be selected from a cognitive deficit, increased hippocampal plaque deposits, and increased neuroinflammation in the brain, relative to a control.

In some embodiments, the mouse does not develop a tumor (does not have a measurable tumor burden).

Some aspects of the present disclosure provide an immunocompromised mouse comprising a nucleic acid encoding a human or humanized APP, wherein the mouse does not have a measurable tumor burden.

Other aspects of the present disclosure provide an immunocompromised mouse comprising a nucleic acid encoding a human or humanized APP, wherein the mouse is at least a year old (e.g., at least 12, 18, or 24 months old).

Yet other aspects of the present disclosure provide a non-obese diabetic (NOD) mouse comprising in its genome a Prkdc^{scid} mutation, an Il2rg^{tm1Wjl} mutation, an APPswe transgene, and a PSEN1de9 transgene.

Also provided herein, in some aspects, is a cell from the mouse of any one of the preceding paragraphs.

Further provided herein, in some aspects, is a mouse comprising a cell having the same genotype of a cell from the mouse of any one of the preceding paragraphs.

A progeny mouse of the mouse of any one of the preceding paragraphs is also provided herein, in some aspects.

Some aspects of the present disclosure provide a method comprising producing the mouse of any one of the preceding paragraphs.

Other aspects of the present disclosure provide a method, comprising introducing into non-obese diabetic (NOD) mouse a null mutation in a murine Prkdc gene, a null mutation in a murine Il2rg gene, a nucleic acid encoding a human or humanized amyloid precursor protein (APP), and a nucleic encoding a mutated human presenilin 1 protein (PSEN1).

Still other aspects of the present disclosure provide a method, comprising introducing into non-obese diabetic (NOD) mouse a Prkdc^{scid} mutation, an Il2rg^{tm1Wjl} mutation, an APPswe transgene, and a PSEN1 de9 transgene.

Further aspects of the present disclosure provide a method, comprising breeding (a) an NOD mouse comprising (i) a loss-of-function mutation in the murine Prkdc gene and (ii) a loss-of-function mutation in a murine Il2rg gene to (b) an NOD mouse comprising (i) a nucleic acid encoding a human or humanized APP and (ii) a nucleic acid encoding a mutated human PSEN1, to produce an immunocompromised progeny mouse having characteristics of early-onset Alzheimer's disease.

Further still, the present disclosure provides, in some aspects, a method, comprising breeding (a) a non-obese diabetic (NOD) mouse comprising a Prkdc^{scid} mutation and a Il2rg^{tm1Wjl} mutation to (b) a NOD mouse comprising an APPswe transgene and a PSEN1de9 transgene.

### BRIEF DESCRIPTION OF THE DRAWINGS

FIG. 1 shows graphs depicting the results of a cognition assessment of both male and female NSG.APP/PSEN1 mice at 7 months on a short-term memory Y-maze task, Novel Spatial Recognition. Intact short-term memory is indicated if the animal spends a higher percentage of time in the novel arm.

FIG. 2 shows immunofluorescent images depicting the results of an amyloid deposition assessment using 1% Thioflavin S stain (diluted in a 1:1 water:ethanol ratio). The images revealed that plaques were primarily limited to the hippocampus, with minimal cortical deposits in the NSG.APP/PSEN1 mouse.

FIG. 3 shows immunofluorescent images depicting the results of staining with markers of neuroinflammation (e.g., microglia activation and astrocyte reactivity), which demonstrate that despite impaired adaptive immunity, NSG.APP/PSEN1 still exhibit robust neuroinflammation in the brain in response to amyloid.

### DETAILED DESCRIPTION

Alzheimer's disease (AD) is the most common cause of dementia. AD affects 35 million people today and its worldwide prevalence is expected to reach 115 million by 2050 due to aging of the population. AD progresses through three stages: preclinical, mild cognitive impairment (MCI), and dementia. Humans with MCI have cognitive deficits but no functional impairments, while humans with dementia exhibit a decline of two or more cognitive domains, which has gradually progressed to the point that functioning at work or daily activities is impaired. Pathologically, AD diagnosis in humans is based on protein aggregates in the brain including amyloid plaques composed of amyloid-beta (Aβ) peptides and neurofibrillary tangles (NFTs) composed of hyperphosphorylated tau. In humans, early spatial distribution of plaque pathology, including plaque pathology occurring first in the hippocampus, correlates strongly with diagnosis of dementia.

Mouse models of AD are limited in that none of the existing models have exhibited the full range of clinical and pathological features of AD, including cognitive and behavioral deficits, amyloid plaques, neurofibrillary tangles, gliosis, synapse loss, axonopathy, neuron loss and neurodegeneration (Hall et al. 2012). Importantly, different mouse models provide varying degrees of AD phenotypes. For example, phenotypes such as cognitive deficits and amyloid plagues are observed in almost all of the mouse models of AD, however human pathology of AD has yet to be recapitulated. In a B6.APP/PSEN1 mouse model, for example, hippocampal and robust cortical plaque deposition is seen at an early timepoint, which is in contrast to human pathology in which plaques are primarily limited to the hippocampus. Unlike the B6.APP/PSEN1 mouse model, the mouse models of the present disclosure, model of AD on an immunodeficient background, exhibits similar amyloid plaque depositions in the hippocampus with minimal cortical deposits, which more closely resembles the human AD pathology. Furthermore, the mouse models of the present disclosure permit a platform for studying immune interactions with amyloid, offering insight to how reduced immunity impacts short-term memory and/or impacts cognitive deficits.

In some embodiments, the present disclosure provides immunodeficient mouse models (e.g., non-obese diabetic (NOD), such as NOD.Cg-Prkdc^{scid} Il2rg^{tm1Wjl}/SzJ (NSG™) mouse models) that comprise a human or humanized amyloid precursor protein (APP) and, in some embodiments, a mutated human presenilin 1 protein (PSEN1).

### Amyloid Precursor Protein

Amyloid precursor protein is a single-pass (type-I) transmembrane precursor protein that is a cleaved into amyloid beta (Aβ), the primary component of amyloid plaques, and is associated with early-onset Alzheimer's disease. Knocking-in chimeric mouse/human amyloid precursor protein can lead to secretion of human amyloid-β (Aβ) peptide. In some embodiments, a mouse model comprises a chimeric nucleic acid that comprises a human coding sequence in the A-beta domain of a mouse APP coding sequence. In some embodiments, the chimeric nucleic acid encodes human Swedish mutations K595N and M596L, relative to a human APP comprising the amino acid sequence of SEQ ID NO: 1. The included Swedish mutations (K595N and M596L) elevate the amount of A-beta produced from the transgene by favoring processing through the beta-secretase pathway (Shin et al. 2010). In some embodiments, the chimeric nucleic acid is the APPswe transgene, which encodes a chimeric amyloid beta (A4) precursor protein comprising the Swedish mutations K595N and M596L (JAX Stock No. 025970).

(SEQ ID NO: 1)
MLPGLALLLLAAWTARALEVPTDGNAGLLAEPQIAMFCGRLNMHMNVQNG
KWDSDPSGTKTCIDTKEGILQYCQEVYPELQITNVVEANQPVTIQNWCKR
GRKQCKTHPHFVIPYRCLVGEFVSDALLVPDKCKFLHQERMDVCETHLHW
HTVAKETCSEKSTNLHDYGMLLPCGIDKFRGVEFVCCPLAEESDNVDSAD
AEEDDSDVWWGGADTDYADGSEDKVVEVAEEEEVAEVEEEEADDDEDDED
GDEVEEEAEEPYEEATERTTSIATTTTTTTESVEEVVREVCSEQAETGPC
RAMISRWYFDVTEGKCAPFFYGGCGGNRNNFDTEEYCMAVCGSAMSQSLL
KTTQEPLARDPVKLPTTAASTPDAVDKYLETPGDENEHAHFQKAKERLEA
KHRERMSQVMREWEEAERQAKNLPKADKKAVIQHFQEKVESLEQEAANER
QQLVETHMARVEAMLNDRRRLALENYITALQAVPPRPRHVFNMLKKYVRA
EQKDRQHTLKHFEHVRMVDPKKAAQIRSQVMTHLRVIYERMNQSLSLLYN
VPAVAEEIQDEVDELLQKEQNYSDDVLANMISEPRISYGNDALMPSLTET
KTTVELLPVNGEFSLDDLQPWHSFGADSVPANTENEVEPVDARPAADRGL
TTRPGSGLTNIKTEEISEVKMDAEFRHDSGYEVHHQKLVFFAEDVGSNKG
AIIGLMVGGVVIATVIVITLVMLKKKQYTSIHHGVVEVDAAVTPEERHLS
KMQQNGYENPTYKFFEQMQN.

### Presenilin 1

Presenilin 1 PSEN1 is a subunit of gamma- (γ-) secretase complex that is involved in the cleavage of APP resulting in the amyloid-β peptide. Mouse models that express mutated human presenilin 1 and a human or humanized APP transgene are associated with early-onset Alzheimer's disease. In some embodiments, a nucleic acid encoding a mutated PSEN1 comprises a human PSEN1 coding sequence that comprises a deletion in exon 9 (DeltaE9) (JAX Stock No. 025970). In some embodiments, the nucleic acid is the PSEN1de9 transgene. In some embodiments, the PSEN1de9 transgene is the Tg(APPswe,PSEN1de9)85Dbo transgene insertion (JAX Stock No. 025970).

### Assessment of Neuroinflammation and Other Symptoms of AD

The APP/PSEN1mouse models of the present disclosure have impaired adaptive immunity. Surprisingly, in some embodiments, an APP/PSEN1 mouse model of the present disclosure has intact innate immune signaling, and thus may be used to assess immune interactions with amyloid through the introduction of material derived from a different strain background or having a human origin. In some embodiments, material derived from a different strain background or having a human origin may include glial cells isolated from a first subject for engraftment in a second subject. As used herein, glial cells may refer to oligodendrocytes, astrocytes, ependymal cells, and/or microglia.

In some embodiments, material derived from a different strain background may include glial cells isolated from mouse models of other backgrounds with intact adaptive immunity. For example, material may be derived from models of other backgrounds, such as the WSB.APP/PSEN1 mouse (JAX Strain No. 025970) or the PWK.APP/PSEN1 mouse (JAX Strain No. 025971), which are non-immunodeficient mouse models. In some embodiments, an APP/PSEN1 mouse model of the present disclosure is used to support engraftment of glial cells isolated from a WSB.APP/PSEN1 mouse model. In some embodiments, an APP/PSEN1 mouse model of the present disclosure is used to support engraftment of glial cells isolated from a PWK.APP/PSEN1 mouse model. In other embodiments, material may be derived from C57BL/6J, 129/S1, A/J, CAST/EiJ, or collaborative lines, or diversity outbred mice.

In some embodiments, material derived from a human origin may include glial cells isolated from human microglia. In some embodiments, an APP/PSEN1 mouse model of the present disclosure is used to support engraftment of glial cells isolated from human microglia.

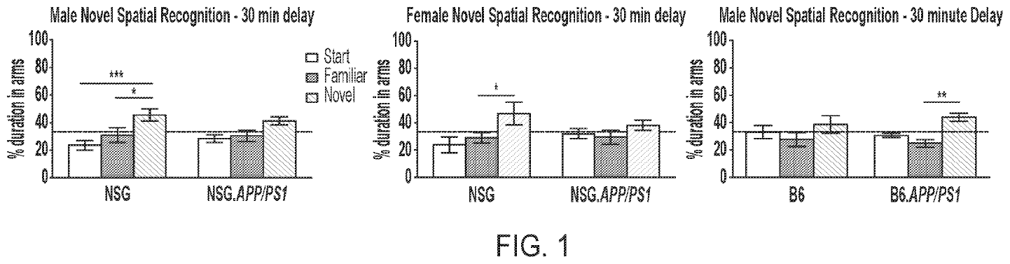

In some embodiments, the APP/PSEN1 mouse model has impaired short-term memory relative to a control mouse (e.g., an NSG® mouse and/or a B6.APP/PSEN1 mouse) of the same age (see, for example, FIG. 1). As used herein, impaired short-term memory refers to changes to the function and structure of neurons in various brain regions. Impaired short-term memory in a mouse may be measured according to, but not limited by, any of the following behavioral assays: delayed alternation, novel spatial recognition, match-to-sample and match-to-place, or contextual fear conditioning.

Delayed alternation refers to tasks that exploit the natural tendency for mice to explore and choose alternate maze arms after re-exposure to the task. The most common delayed alternation task is the Y-maze or T-maze, where the animal begins the task at the stem of the “Y” or “T” and must choose between two arms, one of which has a food reward. Mice with deficits in short-term memory show decreased spontaneous alternation on this task. Novel spatial recognition, a subtype of delayed alternation, refers to a task that exploits the natural tendency for mice to explore novel environments. In some embodiments, an APP/PSEN1 mouse of the present disclosure may show decreased spontaneous alternation and/or decreased novel spatial recognition on this task relative to a control mouse.

Match-to-sample and match-to place tasks require a mouse to remember the identity or location of a stimulus for more than a few seconds. For mice, this task concept is adapted to maze environments, such as delayed non-matched-to-place in the T-maze or water maze. In these tasks, the mouse is cued to make a choice response based on a past representation in order to obtain the escape platform location or a food reward. In some embodiments, an APP/PSEN1 mouse of the present disclosure may have delayed match-to-sample or match-to-place relative to a control mouse.

Contextual Fear Conditioning refers to a task where the mouse is conditioned with an aversive event and then tested for recollection. Mice are usually given a foot shock (unconditioned aversive stimulus) within a specific environment (conditioned neutral stimulus), such that after training the mice will freeze when placed back in the environment. To test short-term memory, the mice are placed in the shock environment up to one hour after training. In some embodiments, an APP/PSEN1 mouse of the present disclosure may show reduced freezing incidences compared to control mice.

Other assays known for measuring impaired short-term memory in rodents are also contemplated herein.

In some embodiments, an APP/PSEN1 mouse model has greater cognitive deficits relative to a control mouse (e.g., an NSG® mouse and/or a B6.APP/PSEN1 mouse) of the same age. As used herein, cognitive deficits are used to describe the impairment of different domains of cognition and is used interchangeably with the term cognitive impairment. Cognitive deficits in a mouse of the present disclosure may be measured according to, but not limited by, any of the following behavioral assays: novel object recognition (NOR), passive inhibitory avoidance, or the Morris water maze task.

The novel object recognition (NOR) task is used to evaluate cognition, particularly recognition memory, in mouse models of CNS disorders. This test is based on the spontaneous tendency of mice to spend more time exploring a novel object than a familiar one. In some embodiments, an APP/PSEN1 mouse of the present disclosure may spend more equal or less time exploring a novel object relative to a familiar object when compared with a control mouse.

The Passive Avoidance task is a fear-aggravated test used to evaluate learning and memory in mouse models of CNS disorders. In this test, mice learn to avoid an environment in which an aversive stimulus (such as a foot-shock) was previously delivered. In some embodiments, an APP/PSEN1 mouse of the present disclosure may not avoid an environment in which an aversive stimulus was previously delivered to said mouse, relative to a control mouse that would avoid an environment in which an aversive stimulus was previously delivered to said mouse.

The Morris water maze is one of the most widely used tasks in behavioral neuroscience for studying the psychological processes and neural mechanisms of spatial learning and memory. Mice are placed in a large circular pool of water and required to escape from water onto a hidden platform whose location can normally be identified only using spatial memory. In some embodiments, an APP/PSEN1 mouse of the present disclosure may not find or may spend a longer time finding the hidden platform relative to a control mouse.

In some embodiments, an APP/PSEN1 mouse model has increased amyloid plaque deposition in the hippocampal region of the brain relative to the cortical region of the brain. As used herein, amyloid plaque deposition refers to the A-beta protein deposition, which accumulates progressively and forms plaque-like lesions throughout the span of the mouse. Amyloid plaque deposition may be measured using immunofluorescent staining of amyloid precursor protein in the cortical and/or hippocampal regions of a mouse brain. Immunofluorescent staining methods, which are well-known in the art, are contemplated herein. Positive staining for amyloid precursor protein, indicating amyloid plaque deposition, may be present in the cortical or the hippocampal region, or both the cortical and hippocampal regions of a mouse brain of the present disclosure. Total immunofluorescent staining of amyloid plaque deposition in the cortical region can be compared relative to the total immunofluorescent staining in the hippocampal region of the same mouse. Total immunofluorescent staining of amyloid plaque deposition in a mouse brain can also be compared relative to the total immunofluorescent staining of amyloid plaque deposition in a control mouse brain.

In some embodiments, in an APP/PSEN1 mouse, the amyloid plaque deposition in the hippocampal region may be increased by at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to the amyloid plaque deposition in the cortical region.

In some embodiments, an APP/PSEN1 mouse of the present disclosure has less cortical plaque deposition relative to the cortical plaque deposition of a control mouse (e.g., an NSG® mouse and/or a B6.APP/PSEN1 mouse) of the same age. As used herein, cortical plaque deposition refers to plaque deposition in the cortical region of the brain. Cortical plaque deposition may be measured using immunofluorescent staining of amyloid precursor protein in the cortical regions of a mouse brain. Immunofluorescent staining methods, which are well-known in the art, are contemplated herein. Positive staining for amyloid precursor protein, indicating cortical plaque deposition, may be present in the cortical regions of a mouse brain of the present disclosure. Total immunofluorescent staining of cortical plaque deposition in a mouse brain is compared relative to the total immunofluorescent staining of cortical plaque deposition in a control mouse brain.

In some embodiments, in an APP/PSEN1 mouse of the present disclosure, the cortical plaque deposition may be decreased by at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to the cortical plaque deposition of a control mouse (e.g., an NSG® mouse and/or a B6.APP/PSEN1 mouse) of the same age.

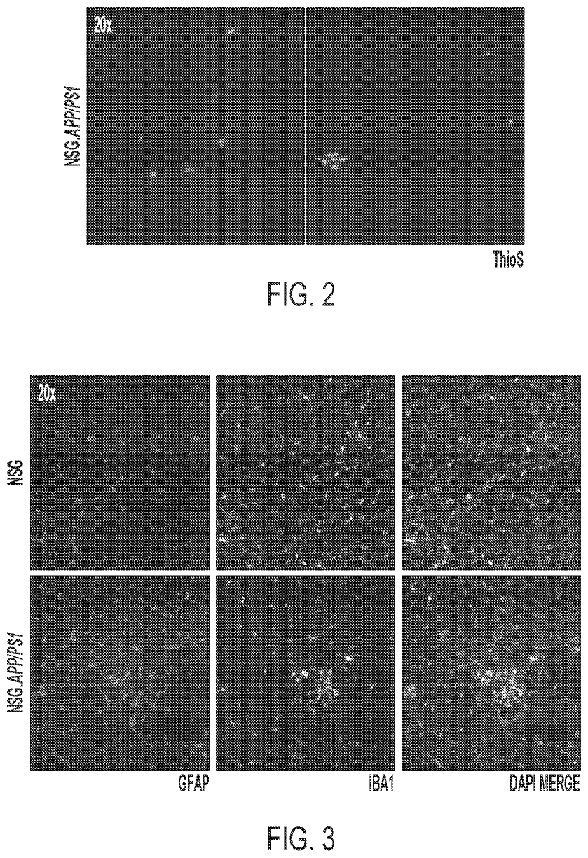

In some embodiments, the plaque region-specificity of an APP/PSEN1 mouse of the present disclosure is different relative to a control mouse (e.g., an NSG® mouse and/or a B6.APP/PSEN1 mouse) of the same age (see, for example, FIG. 2). As used herein, plaque region-specificity refers to the region of the mouse brain (e.g., the cortical or hippocampal region, or both the cortical or hippocampal regions) wherein amyloid plaque deposition may occur. In humans, plaque pathology occurs first in hippocampus (i.e., plaque region-specificity in humans occurs first in the hippocampal region). The B6.APP/PSEN1 mouse model exhibits plaque region-specificity in both the cortical and hippocampal regions of the mouse brain. In some embodiments, the plaque region-specificity of an APP/PSEN1 mouse of the present disclosure develops in a similar way to the plaque region-specificity reported in humans (e.g., wherein plaque pathology occurs first in the hippocampus). Furthermore, NSG® mouse models do not demonstrate plaque pathology.

In some embodiments, the neuroinflammation of an APP/PSEN1 mouse is different relative to a control mouse (e.g., an NSG® mouse and/or a B6.APP/PSEN1 mouse) of the same age (see, for example, FIG. 3). As used herein, neuroinflammation is indicated by positive immunofluorescent staining of microglia activation and astrocyte reactivity in the brain. Microglia activation may be measured by staining brain tissue with markers of microglia. Astrocyte reactivity may be measured by staining brain tissue with markers of astrocytes. Total immunofluorescent staining of microglia activation and/or astrocyte reactivity in the mouse brain can be compared relative to the total immunofluorescent staining microglia activation and/or astrocyte reactivity in a control mouse brain.

In some embodiments, the neuroinflammation (e.g., indicated by immunofluorescent staining of astrocyte reactivity) of an APP/PSEN1 mouse is higher relative to a control mouse (e.g., an NSG® mouse and/or a B6.APP/PSEN1 mouse) of the same age. In some embodiments, the neuroinflammation (e.g., indicated by immunofluorescent staining of astrocyte reactivity) of an APP/PSEN1 mouse may be increased by at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control mouse (e.g., an NSG® mouse and/or a B6.APP/PSEN1 mouse) of the same age.

In some embodiments, the neuroinflammation (e.g., indicated by immunofluorescent staining of microglia activation) of an APP/PSEN1 mouse is higher relative to a control mouse (e.g., an NSG® mouse and/or a B6.APP/PSEN1 mouse) of the same age. In some embodiments, the neuroinflammation (e.g., indicated by immunofluorescent staining of microglia activation) of an APP/PSEN1 mouse may be increased by at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control mouse (e.g., an NSG® mouse and/or a B6.APP/PSEN1 mouse) of the same age.

### Methods of Use

The mouse models provided herein (e.g., the APP/PSEN1 mouse model) may be used for any number of applications. In some embodiments, a mouse model of the present disclosure exhibits robust neuroinflammation in the brain in response to amyloid despite having impaired adaptive immunity, indicating the mouse model has intact innate immune signaling. Therefore, a mouse model of the present disclosure may be used as a platform for the assessment of immune interactions with amyloid through introduction of material derived from different strain backgrounds or human origin as described above.

In some embodiments, a mouse model of the present disclosure may be used to evaluate immune interactions with amyloid in the context of Alzheimer's disease (AD). In some embodiments, the AD may be early onset AD. In some embodiments, a mouse model of the present disclosure may be used to evaluate amyloid plaque deposition, cortical plaque deposition, plaque region-specificity, and/or neuroinflammation as described above in the context of early onset AD.

In some embodiments, a mouse model of the present disclosure may be used to test how a particular agent (e.g., therapeutic agent) or medical procedure (e.g., cell or tissue transplantation) impacts neuroinflammation (e.g., microglia activation and astrocyte reactivity) in response to amyloid. In some embodiments, a particular agent (e.g., therapeutic agent) may be delivered to a mouse model of the present disclosure, and changes in neuroinflammation as a result of said agent may be measured as described above relative to a mouse model of the present disclosure that did not receive said agent. Changes in neuroinflammation as a result of treatment with an agent may be indicated by an increase or decrease in microglial activation and astrocyte reactivity as described above.

Non-limiting examples of agents include therapeutic agents, such as anti-cancer agents and anti-inflammatory agents, and prophylactic agents, such as immunogenic compositions (e.g., vaccines).

In some embodiments, a mouse model of the present disclosure may receive a medical procedure (e.g., cell or tissue transplantation), and changes in neuroinflammation as a result of said medical procedure may be measured as described above relative to a mouse model of the present disclosure that did not receive said medical procedure. Changes in neuroinflammation as a result of the medical procedure (e.g., cell or tissue transplantation) may be indicated by an increase or decrease in microglial activation and astrocyte reactivity as described above.

Non-limiting examples of medical procedures include transplantation of cells (e.g., microglia) from other mouse background strains or from human origin as described above. In some embodiments, a mouse model of the present disclosure may be used to evaluate the effect of transplantation of cells from different genetic backgrounds (e.g., microglia cells isolated from WSB.APP/PSEN1 and/or from PWK.APP/PSEN1) as described above. In some embodiments, a mouse model of the present disclosure may be used to evaluate the effect of transplantation of cells from human microglia as described above. In some embodiments, transplantation from other mouse background strains include, but are not limited to, mouse background strains C57BL/6J, 129/S1, A/J, CAST/EiJ, and diversity outbred (DO) mice. Transplantation from other mouse background strains and other cells of human origin are also contemplated.

In some embodiments, a mouse model of the present disclosure (e.g., the APP/PSEN1 mouse model) may be used to evaluate an effect of an agent or medical procedure on neuroinflammation in response to amyloid. Thus, provided herein are methods that comprise administering an agent or medical procedure to a mouse model, and evaluating an effect of the agent or medical procedure on neuroinflammation in response to amyloid in the mouse.

Assessing an effect of an agent or medical procedure on neuroinflammation in response to amyloid in a mouse model of the present disclosure (e.g., the APP/PSEN1 mouse model) includes, for example, comparing the result of the assessment with a suitable control, such as, but not limited to, the effect of the compound on a control mouse, such as a non-immunodeficient mouse expressing human APP and mutated human PSEN1 (e.g., B6.APP/PSEN1) or a wild-type mouse (e.g., a mouse not expressing human APP and mutated human PSEN1).

In some embodiments, a mouse model of the present disclosure (e.g., the APP/PSEN1 mouse model) may be used to evaluate an effect of an agent or medical procedure on amyloid plaque deposition. Thus, provided herein are methods that comprise administering an agent or medical procedure to a mouse model, and evaluating an effect of the agent or medical procedure on amyloid plaque deposition in the mouse. Changes in amyloid plaque deposition as a result of the agent or medical procedure may be indicated by an increase or decrease in amyloid staining in the cortical and/or the hippocampal regions of the mouse brain as described above. In some embodiments, a decrease in amyloid plaque deposition as a result of the agent or medical procedure may be indicative of a reduction in progression of the AD phenotype in the mouse.

Assessing an effect of an agent or medical procedure on amyloid plaque deposition in a mouse model of the present disclosure (e.g., the APP/PSEN1 mouse model) includes, for example, comparing the result of the assessment with a suitable control, such as, but not limited to, the effect of the compound on a control mouse, such as a non-immunodeficient mouse expressing human APP and mutated human PSEN1 (e.g., B6.APP/PSEN1) or a wild-type mouse (e.g., a mouse not expressing human APP and mutated human PSEN1).

In some embodiments, a mouse model of the present disclosure (e.g., the APP/PSEN1 mouse model) may be used to evaluate an effect of an agent or medical procedure on cortical plaque deposition. Changes in cortical plaque deposition as a result of the agent or medical procedure may be indicated by an increase or decrease in amyloid staining in the cortical region of the mouse brain as described above. In some embodiments, a decrease in amyloid plaque deposition as a result of the agent or medical procedure may be indicative of a reduction in progression of the AD phenotype in the mouse.

Assessing an effect of an agent or medical procedure on cortical plaque deposition in a mouse model of the present disclosure (e.g., the APP/PSEN1 mouse model) includes, for example, comparing the result of the assessment with a suitable control, such as, but not limited to, the effect of the compound on a control mouse, such as a non-immunodeficient mouse expressing human APP and mutated human PSEN1 (e.g., B6.APP/PSEN1) or a wild-type mouse (e.g., a mouse not expressing human APP and mutated human PSEN1).

In some embodiments, a mouse model of the present disclosure (e.g., the APP/PSEN1 mouse model) may be used to evaluate an effect of an agent or medical procedure on plaque region-specificity. Changes in plaque region-specificity as a result of the agent or medical procedure may be indicated by an increase or decrease in amyloid staining in the cortical and/or hippocampal regions of the mouse brain as described above. In some embodiments, a decrease in amyloid plaque deposition in the cortical and/or the hippocampal regions of the mouse brain as a result of the agent or medical procedure may be indicative of a reduction in progression of the AD phenotype in the mouse.

Assessing an effect of an agent or medical procedure on plaque region-specificity in a mouse model of the present disclosure (e.g., the APP/PSEN1 mouse model) includes, for example, comparing the result of the assessment with a suitable control, such as, but not limited to, the effect of the compound on a control mouse, such as a non-immunodeficient mouse expressing human APP and mutated human PSEN1 (e.g., B6.APP/PSEN1) or a wild-type mouse (e.g., a mouse not expressing human APP and mutated human PSEN1).

In some embodiments, a mouse model of the present disclosure (e.g., the APP/PSEN1 mouse model) may be used to evaluate an effect of an agent or medical procedure on short term memory. Changes in short term memory as a result of the agent or medical procedure may be indicated by improved performance in any one of the behavioral assays used to measure short term memory described above. In some embodiments, improved performance in any one of the behavioral assays used to measure short term memory may indicate a reduction in progression of the AD phenotype in the mouse.

Assessing an effect of an agent or medical procedure on short term memory in a mouse model of the present disclosure (e.g., the APP/PSEN1 mouse model) includes, for example, comparing the result of the assessment with a suitable control, such as, but not limited to, the effect of the compound on a control mouse, such as a non-immunodeficient mouse expressing human APP and mutated human PSEN1 (e.g., B6.APP/PSEN1) or a wild-type mouse (e.g., a mouse not expressing human APP and mutated human PSEN1).

In some embodiments, a mouse model of the present disclosure (e.g., the APP/PSEN1 mouse model) may be used to evaluate an effect of an agent or medical procedure on cognitive deficits. Changes in cognitive deficits as a result of the agent or medical procedure may be indicated by an improved performance in any one of the behavioral assays used to measure cognitive deficits described above. In some embodiments, improved performance in any one of the behavioral assays used to measure cognitive deficits may indicate a reduction in progression of the AD phenotype in the mouse.

Assessing an effect of an agent or medical procedure on cognitive deficits in a mouse model of the present disclosure (e.g., the APP/PSEN1 mouse model) includes, for example, comparing the result of the assessment with a suitable control, such as, but not limited to, the effect of the compound on a control mouse, such as a non-immunodeficient mouse expressing human APP and mutated human PSEN1 (e.g., B6.APP/PSEN1) or a wild-type mouse (e.g., a mouse not expressing human APP and mutated human PSEN1).

### Mouse Models

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The registry is maintained at the Institute for Laboratory Animal Research (ILAR) at the National Academy of Sciences, Washington, D.C. Lab Codes may be obtained electronically at ILAR's web site (nationalacademies.org/ilar/institute-for-laboratory-animal-research). See also Davis son MT, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

The mouse models provide herein are transgenic mouse models that express a human or humanized amyloid precursor protein (APP). In some embodiments, the transgenic mouse models also express a human presenilin 1 protein (PSEN1). A transgenic mouse is a mouse having an exogenous nucleic acid (e.g., transgene) in (integrated into) its genome. Methods of producing transgenic mice are well-known.

Three conventional methods used for the production of transgenic mice include DNA microinjection (Gordon and Ruddle, Science 1981: 214: 1244-124, incorporated herein by reference), embryonic stem cell-mediated gene transfer (Gossler et al., Proc. Natl. Acad. Sci. 1986, 83: 9065-9069, incorporated herein by reference) and retrovirus-mediated gene transfer (Jaenisch, Proc. Natl. Acad. Sci. 1976, 73: 1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a nucleic acid encoding human FcRn and/or a chimeric IgG antibody may be confirmed, for example, using any number of genotyping methods (e.g., sequencing and/or genomic PCR).

New mouse models can also be created by breeding parental lines, as described in the Examples herein. With the variety of available mutant, knock-out, knock-in, transgenic, Cre-lox, Tet-inducible system, and other mouse strains, multiple mutations and transgenes may be combined to generate new mouse models. Multiple mouse strains may be bred together to generate double, triple, or even quadruple and higher multiple mutant/transgenic mice.

In some embodiments, parental mice are bred to produce F1 mice. A parental mouse may be, for example, homozygous, heterozygous, hemizygous, or homozygous null at a particular allele. Homozygous describes a genotype of two identical alleles at a given locus, heterozygous describes a genotype of two different alleles at a locus, hemizygous describes a genotype consisting of only a single copy of a particular gene in an otherwise diploid organism, and homozygous null refers to an otherwise-diploid organism in which both copies of the gene are missing.

In some embodiments, an NOD mouse comprising a loss-of-function mutation in the murine Prkdc gene and a loss-of-function mutation in a murine Il2rg gene is bred to an NOD mouse comprising a nucleic acid encoding a human or humanized amyloid precursor protein and a nucleic acid encoding a mutated human presenilin 1 protein, to produce an immunocompromised progeny mouse having characteristics of early-onset Alzheimer's disease. Methods comprising propagating the progeny mice are also contemplated.

In some embodiments, a non-obese diabetic (NOD) mouse comprising a Prkdc^{scid} mutation and an Il2rg^{tm1Wjl} mutation is bred to a NOD mouse comprising an APPswe transgene and a PSENde9 transgene. Methods comprising propagating the progeny mice are also contemplated.

In some embodiments, a male mouse comprising a background of NOD.Cg-Tg(APPswe,PSEN1dE9)85Dbo/How (JAX Stock No. 25967) is bred to a female mouse comprising a background of NOD.Cg-Prkdc^{scid} Il2rg^{tm1Wjl}/SzJ (JAX Stock No. 005557), and the resulting male offspring genotyped for the presence of the APP/PSEN1 transgene and gamma mutation were then subsequently crossed to the female NSG® mice. Methods comprising propagating the progeny mice are also contemplated.

F1 hybrid mice are produced by crossing mice of two different inbred strains. Although they are heterozygous at all loci for which their parents have different alleles, they are similar to inbred strains in that they are genetically and phenotypically uniform. As long as the parental strains exist, F1 hybrids can be generated. However, unlike the parent strains, F1 hybrids do not breed true: the F2 offspring produced by mating F1 mice all have a unique random mixture of alleles from both parental strains.

In some embodiments of the present disclosure, one or more cells may be isolated from a mouse described by the present disclosure. In some embodiments, one or more cells isolated from a mouse of the present disclosure comprise the same genotype of a cell from said mouse.

Immunodeficient Mouse Models

Provided herein, in some embodiments, are immunodeficient mouse models. As is known in the art, immunodeficient mice have impaired or disrupted immune systems, such as specific deficiencies in MHC class I, II or both, B cell or T cell defects, or defects in both, as well as immunodeficiency due to knockdown of genes for cytokines, cytokine receptors, TLR receptors and a variety of transducers and transcription factors of signaling pathways. Immunodeficiency mouse models include the single-gene mutation models such as nude-mice (nu) strains and the severe combined immunodeficiency (scid) strains, non-obese diabetic (NOD) strain, RAG (recombination activating gene) strains with targeted gene deletion and a variety of hybrids originated by crossing doubly and triple mutation mice strains with additional defects in innate and adaptive immunity.

Non-limiting examples of spontaneous and transgenic immunodeficient mouse models include the following mouse strains:
Nude (nu) [Flanagan S P. Genet Res 1966; 8: 295-309; and Nehls M et al. Nature 1994; 372: 103-7];
Scid (scid) [Bosma G C et al. Nature 1983; 301:527-30; Mosier D E et al. Nature 1988; 335: 256-9; and Greiner D L et al. Stem Cells 1998; 16: 166-77];
NOD [Kikutani H et al. Adv Immunol 1992; 51: 285-322; and Anderson M S et al. Ann Rev Immunol 2005; 23: 447-85];
RAG1 and RAG2 (rag) [Mombaerts P et al. Cell 1992; 68: 869-77; Shinkai U et al. Cell 1992; 68: 855-67];
NOD-scid [Greiner D L et al. 1998; Shultz L D et al. J Immunol 1995; 154: 180-91; Melkus M W et al. Nature Med 2006; 12: 1316-22; and Denton P W et al. PLoS Med 2008; 4(12): e357];
IL2rgnull [DiSanto J P et al. Proc Natl Acad Sci USA 1995; 92: 377-81];
B2mnull [Christianson S W et al. J Immunol 1997; 158: 3578-86];
NOD-scid IL2rγnull [Shultz L D et al. Nat Rev Immunol 2007; 7: 118-30; Ito M et al. Blood 2002; 100: 3175-82; Ishikawa I et al. Blood 2005; 106: 1565-73; and Macchiarini F et al. J Exp Med 2005; 202: 1307-11];
NOD-scid B2mnull [Shultz et al. 2007; Shultz L D et al. Transplantation 2003; 76: 1036-42; Islas-Ohlmayer M A et al. J Virol 2004; 78:13891-900; and Macchiarini et al. 2005]; and
HLA transgenic mice [Grusby M J et al. Proc Natl Acad Sci USA 1993; 90(9): 3913-7; and Roy C J et al. Infect Immun 2005; 73(4): 2452-60]. See, e.g., Belizario J E The Open Immunology Journal, 2009; 2:79-85.

Provided herein, in some embodiments, are immunodeficient mouse models having the non-obese diabetic (NOD) mouse genotype. The NOD mouse (e.g., Jackson Labs Stock #001976, NOD-Shi^{LtJ}) is a polygenic mouse model of autoimmune (e.g., Type 1) diabetes, characterized by hyperglycemia and insulitis, a leukocytic infiltration of the pancreatic islet cells. The NOD mice are hypoinsulinemic and hyperglucagonemic, indicating a selective destruction of pancreatic islet beta cells. The major component of diabetes susceptibility in NOD mice is the unique MHC haplotype. NOD mice also exhibit multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. They also lack hemolytic complement, C5. NOD mice also are severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background may have a genotype selected from NOD-Cg.-Prkdc^{scid}IL2rg^{tm1wJl}/SzJ (NSG®), a NOD.Cg-Rag1^{tm1Mom} Il2rg^{tm1Wjl}/SzJ (NRG), and NOD.Cg-Prkdc^{scid}Il2rg^{tm1Sug}/ShiJic (NOG). Other immunodeficient mouse strains are contemplated herein.

In some embodiments, an immunodeficient mouse model has an NSG™ genotype. The NSG® mouse (e.g., Jackson Labs Stock No.: #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG® mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2rg^{tm1Wjl} targeted mutation. The Prkdc^{scid} mutation is a loss-of-function (null) mutation in the mouse homolog of the human PRKDC gene -this mutation essentially eliminates adaptive immunity (see, e.g., (Blunt et al., 1995; Greiner, Hesselton, & Shultz, 1998), each of which is incorporated herein by reference). The Il2rg^{tm1Wjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse model has an NRG genotype. The NRG mouse (e.g., Jackson Labs Stock #007799) is extremely immunodeficient. This mouse comprises two mutations on the NOD/ShiLtJ genetic background; a targeted knockout mutation in recombination activating gene 1 (Rag¹) and a complete null allele of the IL2 receptor common gamma chain (IL2rg^{null}). The Rag1^{null} mutation renders the mice B and T cell deficient and the IL2rg^{null} mutation prevents cytokine signaling through multiple receptors, leading to a deficiency in functional NK cells. The extreme immunodeficiency of NRG allows the mice to be humanized by engraftment of human CD34⁺ hematopoietic stem cells (HSC) and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL-2 receptor-7 chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG genotype. The NCG mouse (e.g., Charles River Stock #572) was created by sequential CRISPR/Cas9 editing of the Prkdc and Il2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

Provided herein, in some embodiments, are immunodeficient mouse models that are deficient in MHC Class I, MHC Class II, or MHC Class I and MHC Class II. A mouse that is deficient in MHC Class I and/or MHC Class II does not express the same level of MHC Class I proteins (e.g., α-microglobulin and β2-microglobulin (B2M)) and/or MHC Class II proteins (e.g., a chain and β chain) or does not have the same level of MHC Class I and/or MHC Class II protein activity as a non-immunodeficient (e.g., MHC Class 1/II wild-type) mouse. In some embodiments, the expression or activity of MHC Class I and/or MI-IC Class II proteins is reduced (e.g., by at least 30%, at least 40%, at least 50%, at least 60%, at least 70%, at least 80%, at least 90% or more), relative to a non-immunodeficient mouse.

Immunodeficient mice that are deficient in MHC Class I, MHC Class II, and MHC Class I and MHC Class II are described in International Publication No. WO 2018/209344, the contents of which are incorporated by reference herein.

Humanized Mouse Models

Provided herein, in some embodiments, are humanized immunodeficient mouse models and methods of producing the models. Immunodeficient mice engrafted with functional human cells and/or tissues are referred to as “humanized mice.” As used herein, the terms “humanized mouse”, “humanized immune deficient mouse”, “humanized immunodeficient mouse”, and the plural versions thereof are used interchangeably to refer to an immunodeficient mouse humanized by engraftment with functional human cells and/or tissues. For example, mouse models may be engrafted with human hematopoietic stem cells (HSCs) and/or human peripheral blood mononuclear cells (PMBCs). In some embodiments, mouse models are engrafted with human tissues such as islets, liver, skin, and/or solid or hematologic cancers. In other embodiments, mouse models may be genetically modified such that endogenous mouse genes are converted to human homologs (see, e.g., Pearson, et al., Curr Protoc Immunol., 2008, Chapter: Unit—15.21).

Humanized mice are generated by starting with an immunodeficient mouse and, if necessary, depleting and/or suppressing any remaining murine immune cells (e.g., chemically or with radiation). That is, successful survival of the human immune system in the immunodeficient mice may require suppression of the mouse's immune system to prevent GVHD (graft-versus-host disease) rejections. After the immunodeficient mouse's immune system has been sufficiently suppressed, the mouse is engrafted with human cells (e.g., HSCs and/or PBMCs). As used herein, “engraft” refers to the process of the human cells migrating to, and incorporating into, an existing tissue of interest in vivo. With respect to the humanized immunodeficient mouse, the engrafted human cells provide functional mature human cells (e.g., immune cells). The model has a specific time window of about 4-5 weeks after engraftment before GVHD sets in. To increase the longevity of the model, double-knockout mice lacking functional MHC I and MHC II, as described above, may be used.

The engrafted human cells (e.g., HSCs or PMBCs) for humanization, in some embodiments, are human leukocyte-antigen (HLA)-matched to the human cancer cells of the mouse models. HLA-matched refers to cells that express the same major histocompatibility complex (MHC) genes. Engrafting mice with HLA-matched human xenografts and human immune cells, for example, reduces or prevents immunogenicity of the human immune cells. In some embodiments, a humanized mouse provided in the present disclosure is engrafted with human PMBCs or human HSCs that are HLA-matched to a PDX or human cancer cell line.

Irradiation

As described above, in some embodiments, immunodeficient mice are irradiated prior to engraftment with human cells, such as human HSCs and/or PMBCs. It is thought that irradiation of an immunodeficient mouse destroys mouse immune cells in peripheral blood, spleen, and bone marrow, which facilitates engraftment of human cells, such as human HSCs and/or PMBCs (e.g., by increasing human cell survival factors), as well as expansion of other immune cells. Irradiation also shortens the time it takes to accumulate the required number of human immune cells to “humanize” the mouse models.

For immunodeficient mice (e.g., NSG® mice), this preparation is commonly accomplished through whole-body gamma irradiation. Irradiators may vary in size depending on their intended use. Animals are generally irradiated for short periods of time (less than 15 min). The amount of time spent inside the irradiator varies depending on the radioisotope decay charts, amount of irradiation needed, and source of ionizing energy (that is, X-rays versus gamma rays, for which a cesium or cobalt source is needed).

A myeloablative irradiation dose is usually 700 to 1300 cGy, though in some embodiments, lower doses such as 1-100 cGy (e.g., about 2, 5, or 10 cGy), or 300-700 cGy may be used.

As an example, the mouse may be irradiated with 100 cGy X-ray (or 75 cGy-125 cGy X-ray). In some embodiments, the dose is about 1, 2, 3, 4, 5, 10, 20, 100, 200, 300, 400, 500, 600, 700, 800, 900, 1000, 1100, 1200, or 1300 cGy, or between any of the two recited doses herein, such as 100-300 cGy, 200-500 cGy, 600-1000 cGy, or 700-1300 cGy. In some embodiments, the immunodeficient mouse is irradiated about 15 minutes, 30 minutes, 45 minutes, 1 hour, or more before engraftment with human HSCs and/or PMBCs. In some embodiments, the immunodeficient mouse is engrafted with human HSCs and/or PMBCs 1, 2, 3, 4, 5, 6, 7, 8, 9, 10, 11, 12, 13, 14, 15, 16, 17, or 18 days after irradiation.

Engraftment

As described above, in some embodiments, the irradiated immunodeficient mice are engrafted with HSCs and/or PBMCs, humanizing the mice. Engraftment refers to the process of the human cells migrating to, and incorporating into, an existing tissue of interest in vivo. The PBMCs may be engrafted after irradiation and before engraftment of human cancer cells, after irradiation and concurrently with engraftment of human cancer cells, or after irradiation and after engraftment of human cancer cells.

Peripheral blood mononuclear cells (PBMCs) are peripheral blood cells having a round nucleus. These mononuclear blood cells recirculate between tissues and blood and are a critical component in the immune system to fight infection and adapt to intruders. There are two main types of mononuclear cells, lymphocytes and monocytes. The lymphocyte population of PBMCs typically includes T cells, B cells and NK cells.

PBMCs may be isolated from whole blood samples, for example (e.g., Ficoll gradient). In some embodiments, PBMCs from a subject (e.g., a human subject) with a current or previous diagnosis of a pathogen or pathogenic disease may be used.

Hematopoietic stem cells (HSCs) are the stem cells that give rise to other blood cells during a process referred to as hematopoiesis. Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells.

Methods of engrafting immunodeficient mice with HSCs and/or PBMCs to yield a humanized mouse model include but are not limited to intraperitoneal or intravenous injection (Shultz et al., J Immunol, 2005, 174:6477-6489; Pearson et al., Curr Protoc Immunol. 2008; 15-21; Kim et al., AIDS Res Hum Retrovirus, 2016, 32(2): 194-202; Yaguchi et al., Cell & Mol Immunol, 2018, 15:953-962). In some embodiments, the mouse is engrafted with 1.0×10⁶3.0×10⁷ HSCs and/or PBMCs.

For example, the mouse may be engrafted with 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.5×10⁶, 3.0×10⁶ or more HSCs and/or PBMCs. In some embodiments, the mouse is engrafted with 1.0-1.1×10⁶, 1.0-1.2×10⁶, 1.0-1.3×10⁶, 1.0-1.4×10⁶, 1.0-1.5×10⁶, 1.0-1.6×10⁶, 1.0-1.7×10⁶, 1.0-1.8×10⁶, 1.0-1.9×10⁶, 1.0-2.0×10⁶, 1.0-2.25×10⁶, 1.0-2.5×10⁶, 1.0-2.75×10⁶, 1.0-3.0×10⁶, 1.1-1.2×10⁶, 1.1-1.3×10⁶, 1.1-1.4×10⁶, 1.1-1.5×10⁶, 1.1-1.6×10⁶, 1.1-1.7×10⁶, 1.1-1.8×10⁶, 1.1-1.9×10⁶, 1.1-2.0×10⁶, 1.1-2.25×10⁶, 1.1-2.5×10⁶, 1.1-2.75×10⁶, 1.1-3.0×10⁶, 1.2-1.3×10⁶, 1.2-1.4×10⁶, 1.2-1.5×10⁶, 1.2-1.6×10⁶, 1.2-1.7×10⁶, 1.2-1.8×10⁶, 1.2-1.9×10⁶, 1.2-2.0×10⁶, 1.2-2.25×10⁶, 1.2-2.5×10⁶, 1.2-2.75×10⁶, 1.2-3.0×10⁶, 1.3-1.4×10⁶, 1.3-1.5×10⁶, 1.3-1.6×10⁶, 1.3-1.7×10⁶, 1.3-1.8×10⁶, 1.3-1.9×10⁶, 1.3-2.0×10⁶, 1.3-2.25×10⁶, 1.3-2.5×10⁶, 1.3-2.75×10⁶, 1.3-3.0×10⁶, 1.4-1.5×10⁶, 1.4-1.6×10⁶, 1.4-1.7×10⁶, 1.4-1.8×10⁶, 1.4-1.9×10⁶, 1.4-2.0×10⁶, 1.4-2.25×10⁶, 1.4-2.5×10⁶, 1.4-2.75×10⁶, 1.4-3.0×10⁶, 1.5-1.6×10⁶, 1.5-1.7×10⁶, 1.5-1.8×10⁶, 1.5-1.9×10⁶, 1.5-2.0×10⁶, 1.5-2.25×10⁶, 1.5-2.5×10⁶, 1.5-2.75×10⁶, 1.5-3.0×10⁶, 1.6-1.7×10⁶, 1.6-1.8×10⁶, 1.6-1.9×10⁶, 1.6-2.0×10⁶, 1.6-2.25×10⁶, 1.6-2.5×10⁶, 1.6-2.75×10⁶, 1.6-3.0×10⁶, 1.7-1.8×10⁶, 1.7-1.9×10⁶, 1.7-2.0×10⁶, 1.7-2.25×10⁶, 1.7-2.5×10⁶, 1.7-2.75×10⁶, 1.7-3.0×10⁶, 1.8-1.9×10⁶, 1.8-2.0×10⁶, 1.8-2.25×10⁶, 1.8-2.5×10⁶, 1.8-2.75×10⁶, 1.8-3.0×10⁶, 1.9-2.0×10⁶, 1.9-2.25×10⁶, 1.9-2.5×10⁶, 1.9-2.75×10⁶, 1.9-3.0×10⁶, 2.0-2.25×10⁶, 2.0-2.5×10⁶, 2.0-2.75×10⁶, 2.0-3.0×10⁶, 2.25-2.5×10⁶, 2.25-2.75×10⁶, 2.25-3.0×10⁶, 2.5-2.75×10⁶, 2.5-3.0×10⁶, or 2.75-3.0×10⁶HSCs and/or PBMCs.

In some embodiments, the mouse may be engrafted with 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.5×10⁷, 3.0×10⁷ or more HSCs and/or PBMCs. In some embodiments, the mouse is engrafted with 1.0-1.1×10⁷, 1.0-1.2×10⁷, 1.0-1.3×10⁷, 1.0-1.4×10⁷, 1.0-1.5×10⁷, 1.0-1.6×10⁷, 1.0-1.7×10⁷, 1.0-1.8×10⁷, 1.0-1.9×10⁷, 1.0-2.0×10⁷, 1.0-2.25×10⁷, 1.0-2.5×10⁷, 1.0-2.75×10⁷, 1.0-3.0×10⁷, 1.1-1.2×10⁷, 1.1-1.3×10⁷, 1.1-1.4×10⁷, 1.1-1.5×10⁷, 1.1-1.6×10⁷, 1.1-1.7×10⁷, 1.1-1.8×10⁷, 1.1-1.9×10⁷, 1.1-2.0×10⁷, 1.1-2.25×10⁷, 1.1-2.5×10⁷, 1.1-2.75×10⁷, 1.1-3.0×10⁷, 1.2-1.3×10⁷, 1.2-1.4×10⁷, 1.2-1.5×10⁷, 1.2-1.6×10⁷, 1.2-1.7×10⁷, 1.2-1.8×10⁷, 1.2-1.9×10⁷, 1.2-2.0×10⁷, 1.2-2.25×10⁷, 1.2-2.5×10⁷, 1.2-2.75×10⁷, 1.2-3.0×10⁷, 1.3-1.4×10⁷, 1.3-1.5×10⁷, 1.3-1.6×10⁷, 1.3-1.7×10⁷, 1.3-1.8×10⁷, 1.3-1.9×10⁷, 1.3-2.0×10⁷, 1.3-2.25×10⁷, 1.3-2.5×10⁷, 1.3-2.75×10⁷, 1.3-3.0×10⁷, 1.4-1.5×10⁷, 1.4-1.6×10⁷, 1.4-1.7×10⁷, 1.4-1.8×10⁷, 1.4-1.9×10⁷, 1.4-2.0×10⁷, 1.4-2.25×10⁷, 1.4-2.5×10⁷, 1.4-2.75×10⁷, 1.4-3.0×10⁷, 1.5-1.6×10⁷, 1.5-1.7×10⁷, 1.5-1.8×10⁷, 1.5-1.9×10⁷, 1.5-2.0×10⁷, 1.5-2.25×10⁷, 1.5-2.5×10⁷, 1.5-2.75×10⁷, 1.5-3.0×10⁷, 1.6-1.7×10⁷, 1.6-1.8×10⁷, 1.6-1.9×10⁷, 1.6-2.0×10⁷, 1.6-2.25×10⁷, 1.6-2.5×10⁷, 1.6-2.75×10⁷, 1.6-3.0×10⁷, 1.7-1.8×10⁷, 1.7-1.9×10⁷, 1.7-2.0×10⁷, 1.7-2.25×10⁷, 1.7-2.5×10⁷, 1.7-2.75×10⁷, 1.7-3.0×10⁷, 1.8-1.9×10⁷, 1.8-2.0×10⁷, 1.8-2.25×10⁷, 1.8-2.5×10⁷, 1.8-2.75×10⁷, 1.8-3.0×10⁷, 1.9-2.0×10⁷, 1.9-2.25×10⁷, 1.9-2.5×10⁷, 1.9-2.75×10⁷, 1.9-3.0×10⁷, 2.0-2.25×10⁷, 2.0-2.5×10⁷, 2.0-2.75×10⁷, 2.0 3.0×10⁷, 2.25-2.5×10⁷, 2.25-2.75×10⁷, 2.25-3.0×10⁷, 2.5-2.75×10⁷, 2.5-3.0×10⁷, or 2.75-3.0×10⁷ HSCs and/or PBMCs. In some embodiments, the mouse is engrafted with 2×10⁷ HSCs and/or PBMCs. According to some embodiments, the mouse is engrafted with 4.5-5.5×10⁷ (4.5 5.0×10⁷, 5.0-5.5×10⁷) HSCs and/or PBMCs.

### Nucleic Acids: Engineering and Delivery

The mouse models described herein comprises a nucleic acid encoding a human or humanized APP and, in some embodiments, a nucleic acid encoding a mutated human PSEN1. In some embodiments, the mouse models described herein also comprise a mouse App allele and/or a mouse Psen1 allele. In some embodiments, the mouse models comprise a human or humanized APP transgene and a mutated human PSEN1 transgene. In some embodiments, a transgene, such as a human APP transgene, and/or a mutated human PSEN1 transgene, is integrated into a mouse genome. Human or humanized APP and mutated human PSEN1 transgenes are described (JAX Stock No. 025970) and incorporated by reference herein.

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome (e.g., in a coding region, non-coding region, and/or regulatory region), relative to the corresponding wild-type nucleic acid (e.g., the naturally-occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A loss-of-function mutation, as is known in the art, results in a gene product with little or no function. A null mutation, which is a type of loss-of-function mutation, results in a gene product with no function. In some embodiments, an inactivated allele is a null allele. Other examples of loss-of-function mutations includes missense mutations and frameshift mutations.

A nucleic acid, such as an allele or alleles of a gene, may be modified such that it does not produce a detectable level of a functional gene product (e.g., a functional protein). Thus, an inactivated allele is an allele that does not produce a detectable level of a functional gene product (e.g., a functional protein). A detectable level of a protein is any level of protein detected using a standard protein detection assay, such as flow cytometry and/or an ELISA. In some embodiments, an inactivated allele is not transcribed. In some embodiments, an inactivated allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example.

Methods for delivering nucleic acids to mouse embryos for the production of transgenic mice include, but are not limited to, electroporation (see, e.g., Wang W et al. J Genet Genomics 2016; 43(5):319-27; WO 2016/054032; and WO 2017/124086, each of which is incorporated herein by reference), DNA microinjection (see, e.g., Gordon and Ruddle, Science 1981: 214: 1244-124, incorporated herein by reference), embryonic stem cell-mediated gene transfer (see, e.g., Gossler et al., Proc. Natl. Acad. Sci. 1986; 83: 9065-9069, incorporated herein by reference), and retrovirus-mediated gene transfer (see, e.g., Jaenisch, Proc. Natl. Acad. Sci. 1976; 73: 1260-1264, incorporated herein by reference), any of which may be used as provided herein.

### Genomic Editing Methods

Engineered nucleic acids, such as guide RNAs, donor polynucleotides, and other nucleic acid coding sequences, for example, may be introduced to a genome of an embryo or cell (e.g., stem cell) using any suitable method. The present application contemplates the use of a variety of gene editing technologies, for example, to introduce nucleic acids into the genome of an embryo or cell to produce a transgenic rodent. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405, each of which is incorporated by reference herein.

In some embodiments, a CRISPR system is used to edit the genome of mouse embryos provided herein. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein, one or multiple guide RNAs (gRNAs), and/or a donor nucleic acid can be delivered, e.g., injected or electroporated, directly into mouse embryos at the one-cell (zygote) stage or a later stage to facilitate homology directed repair (HDR), for example, to introduce an engineered nucleic acid (e.g., donor nucleic acid) into the genome.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitides (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337: 816-821 and Deltcheva et al., Nature, 2011; 471: 602-607, each of which is incorporated by reference herein.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/41. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μ1.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

A donor nucleic acid typically includes a sequence of interest flanked by homology arms. Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

### EXAMPLES

### Example 1. Generation of NSG.APP/PSEN1 Mouse Model

NOD.Cg-Prkdc^{scid}Il2rg^{tm1Wjl}Tg(APPswe, PSEN1dE9)85Dbo/How (JR#29513)(NSG.APP/PSEN1) were generated by crossing male NOD.Cg-Tg(APPswe,PSEN1dE9)85Dbo/How (JR #25967) to female NOD.Cg-Prkdc^{scid}Il2rg^{tm1Wjl}/SzJ (JR #005557). Male offspring were genotyped for the presence of the APP/PSEN1 transgene and gamma mutation and then subsequently crossed to female NSG mice. A cohort of male and female NSG.APP/PSEN1 were generated at N11 for assessment. All mice were maintained on Sulfatrim antibiotic water. These mice represent a unique platform for assessment of immune interactions with amyloid through introduction of material derived from different strain background or human origin.

### Behavioral Phenotype

Cognition of the NSG.APP/PSEN1 mouse model was assessed at 7 months on a short-term memory Y-maze task, Novel Spatial Recognition as previously described (Sukoff Rizzo, 2018). Animals were placed in a Y-maze in which visual cues were placed at the end of each arm. Animals explored the maze for 10 minutes, with one of the arms (novel arm) blocked. After a delay of 30 minutes, animals were re-introduced to the maze for 5 minutes with all arms available. The results are shown in FIG. 1. Intact short-term memory is indicated if the animal spends a higher percentage of time in the novel arm. While both male and female NSG animals exhibited intact short-term memory, transgenic NSG.APP/PSEN1 littermates failed this task and spend an equal percentage of time exploring all the arms. These findings are in contrast relative to the B6.APP/PSEN1 mouse model, which do not exhibit cognitive deficits in this task at 7 months. Furthermore, the B6.APP/PSEN1 mice are not reported to show deficits on other cognitive tasks like the Morris Watermaze until 10 months (AlzForum). These results highlight the utility of the NSG.APP/PSEN1 strain for assessment of immune-related interventions that may prevent cognitive deficits.

### Plaque Deposition

Animals were sacrificed at 8 months via cardiac perfusions with 1×PBS, brains fixed overnight with 4% paraformaldehyde, placed in 15% and then 30% sucrose for 24 hours, blocked in OCT and cryosectioned at 25 microns for assessment of neuropathology. Amyloid deposition was assessed using 1% Thioflavin S stain (diluted in a 1:1 water: ethanol ratio) which revealed that plaques were primarily limited to the hippocampus, with minimal cortical deposits (see FIG. 2). These results suggest a direct link to the short-term memory deficits as this behavioral task requires the hippocampus. This plaque region-specificity is in stark contrast to what is seen in the B6.APP/PSEN1 mouse model, which exhibits hippocampal and robust cortical plague deposition at this timepoint (Jackson et al., 2013; Onos et al., 2019). These results also match what is seen in human patients, in which plaque pathology occurs first in hippocampus. As expected, non-transgenic NSG littermates did not demonstrate plaque pathology.

### Glial Neuroinflammation

Immunofluorescent staining with markers of astrocytes (anti-chicken GFAP, ACRIS/Origene, AP31806PU-N, 1:300) and microglia (anti-rabbit IBA1, Wako, 019-19741, 1:300) demonstrate that despite impaired adaptive immunity, NSG.APP/PSEN1 mice still exhibit robust neuroinflammation in the brain in response to amyloid (see FIG. 3). These results suggest the NS G.APP/PSEN1 mice retain intact innate immune signaling. In view of these findings, NSG.APP/PSEN1 mouse model may be useful for analyzing the effects of transplanted glia from other strain backgrounds or of human origin on cognitive deficiency.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

### REFERENCES

1. Drummond E, Wisniewski T. Alzheimer's disease: experimental models and reality. Acta Neuropathol. 2017 February; 133(2):155-175. doi: 10.1007/s00401-016-1662-x. Epub 2016 Dec. 26. PMID: 28025715; PMCID: PMC5253109.
2. Espuny-Camacho I, Arranz A M, Fiers M, Snellinx A, Ando K, Munck S, Bonnefont J, Lambot L, Corthout N, Omodho L, Vanden Eynden E, Radaelli E, Tesseur I, Wray S, Ebneth A, Hardy J, Leroy K, Brion J P, Vanderhaeghen P, De Strooper B. Hallmarks of Alzheimer's Disease in Stem-Cell-Derived Human Neurons Transplanted into Mouse Brain. Neuron. 2017 Mar. 8; 93(5):1066-1081.e8. doi: 10.1016/j.neuron.2017.02.001. Epub 2017 Feb. 23. PMID: 28238547.
3. Hall A M, Roberson E D. Mouse models of Alzheimer's disease. Brain Res Bull. 2012 May 1; 88(1):3-12. doi: 10.1016/j.brainresbull.2011.11.017. Epub 2011 Nov. 28. PMID: 22142973; PMCID: PMC3546481.
4. Shin J, Yu S B, Yu U Y, Jo S A, Ahn J H. Swedish mutation within amyloid precursor protein modulates global gene expression towards the pathogenesis of Alzheimer's disease. BMB Rep. 2010 October; 43(10):704-9. doi: 10.5483/BMBRep.2010.43.10.704. PMID: 21034535.
5. Mouse Genome Informatics Database. <http://www.informatics.jax.org/>. Last updated 2020 Dec. 8.
6. International Committee on Standardized Genetic Nomenclature for Mice (1996 April) Rules and guidelines for gene nomenclature. In: Lyon, M.F., Rastan, S. and Searle, A.G. eds. Genetic Variants and Strains of The Laboratory Mouse, 3rd Ed. Oxford, UK: Oxford University Press, pp. 1-16.
7. Institute for Laboratory Animal Research. <https://www.nationalacademies.org/ilar/institute-for-laboratory-animal-research>.
8. Davisson M T. Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.
9. Gordon J W, Ruddle F H. Integration and stable germ line transmission of genes injected into mouse pronuclei. Science. 1981 December; 214(4526):1244-6.
10. Gossler A, Doetschman T, Korn R, Serfling E, Kemler R. Transgenesis by means of blastocyst-derived embryonic stem cell lines. Proc Natl Acad Sci USA. 1986 December; 83(23):9065-9. doi: 10.1073/pnas.83.23.9065. PMID: 3024164; PMCID: PMC387075.
11. Jaenisch R. Germ line integration and Mendelian transmission of the exogenous Moloney leukemia virus. Proc Natl Acad Sci USA. 1976 April; 73(4):1260-4. doi: 10.1073/pnas.73.4.1260. PMID: 1063407; PMCID: PMC430242.
12. Flanagan S P. ‘Nude’, a new hairless gene with pleiotropic effects in the mouse. Genet Res. 1966 December; 8(3):295-309. doi: 10.1017/s0016672300010168. PMID: 5980117.
13. Nehls M, Pfeifer D, Schorpp M, Hedrich H, Boehm T. New member of the winged-helix protein family disrupted in mouse and rat nude mutations. Nature. 1994 Nov. 3; 372(6501):103-7. doi: 10.1038/372103a0. PMID: 7969402.
14. Bosma G C, Custer R P, Bosma M J. A severe combined immunodeficiency mutation in the mouse. Nature. 1983 Feb. 10; 301(5900):527-30. doi: 10.1038/301527a0. PMID: 6823332.
15. Mosier D E, Gulizia R J, Baird S M, Wilson D B. Transfer of a functional human immune system to mice with severe combined immunodeficiency. Nature. 1988 Sep. 15; 335(6187):256-9. doi: 10.1038/335256a0. PMID: 2970594.
16. Greiner D L, Hesselton R A, Shultz L D. SCID mouse models of human stem cell engraftment. Stem Cells. 1998; 16(3):166-77. doi: 10.1002/stem.160166. PMID: 9617892.
17. Kikutani H, Makino S. The murine autoimmune diabetes model: NOD and related strains. Adv Immunol. 1992; 51:285-322. doi: 10.1016/s0065-2776(08)60490-3. PMID: 1323922.
18. Anderson M S, Bluestone J A. The NOD mouse: a model of immune dysregulation. Annu Rev Immunol. 2005; 23:447-85. doi: 10.1146/annurev.immunol.23.021704.115643. PMID: 15771578.
19. Mombaerts P, Iacomini J, Johnson R S, Herrup K, Tonegawa S, Papaioannou V E. RAG-1-deficient mice have no mature B and T lymphocytes. Cell. 1992 Mar. 6; 68(5):869-77. doi: 10.1016/0092-8674(92)90030-g. PMID: 1547488.
20. Shinkai Y, Rathbun G, Lam K P, Oltz E M, Stewart V, Mendelsohn M, Charron J, Datta M, Young F, Stall A M, et al. RAG-2-deficient mice lack mature lymphocytes owing to inability to initiate V(D)J rearrangement. Cell. 1992 Mar. 6; 68(5):855-67. doi: 10.1016/0092-8674(92)90029-c. PMID: 1547487.
21. Shultz L D, Schweitzer P A, Christianson S W, Gott B, Schweitzer I B, Tennent B, McKenna S, Mobraaten L, Rajan T V, Greiner D L, et al. Multiple defects in innate and adaptive immunologic function in NOD/LtSz-scid mice. J Immunol. 1995 Jan. 1; 154(1):180-91. PMID: 7995938.
22. Melkus M W, Estes J D, Padgett-Thomas A, Gatlin J, Denton P W, Othieno F A, Wege A K, Haase A T, Garcia J V. Humanized mice mount specific adaptive and innate immune responses to EBV and TSST-1. Nat Med. 2006 November; 12(11):1316-22. doi: 10.1038/nm1431. Epub 2006 Oct. 22. PMID: 17057712.
23. Denton P W, Estes J D, Sun Z, Othieno F A, Wei B L, Wege A K, Powell D A, Payne D, Haase A T, Garcia J V. Antiretroviral Pre-exposure Prophylaxis Prevents Vaginal Transmission of HIV-1 in Humanized BLT Mice. PLoS Med 2008; 5(1):e16.
24. DiSanto J P, Müller W, Guy-Grand D, Fischer A, Rajewsky K. Lymphoid development in mice with a targeted deletion of the interleukin 2 receptor gamma chain. Proc Natl Acad Sci USA. 1995 Jan. 17; 92(2):377-81. doi: 10.1073/pnas.92.2.377. PMID: 7831294; PMCID: PMC42743.
25. Christianson S W, Greiner D L, Hesselton R A, Leif J H, Wagar E J, Schweitzer I B, Rajan T V, Gott B, Roopenian D C, Shultz L D. Enhanced human CD4+ T cell engraftment in beta2-microglobulin-deficient NOD-scid mice. J Immunol. 1997 Apr. 15; 158(8):3578-86. PMID: 9103418.
26. Shultz L D, Ishikawa F, Greiner D L. Humanized mice in translational biomedical research. Nat Rev Immunol. 2007 February; 7(2):118-30. doi: 10.1038/nri2017. PMID: 17259968.
27. Ito M, Hiramatsu H, Kobayashi K, Suzue K, Kawahata M, Hioki K, Ueyama Y, Koyanagi Y, Sugamura K, Tsuji K, Heike T, Nakahata T. NOD/SCID/gamma(c)(null) mouse: an excellent recipient mouse model for engraftment of human cells. Blood. 2002 Nov. 1; 100(9):3175-82. doi: 10.1182/blood-2001-12-0207. PMID: 12384415.
28. Ishikawa F, Yasukawa M, Lyons B, Yoshida S, Miyamoto T, Yoshimoto G, Watanabe T, Akashi K, Shultz L D, Harada M. Development of functional human blood and immune systems in NOD/SCID/IL2 receptor {gamma} chain(null) mice. Blood. 2005 Sep. 1; 106(5):1565-73. doi: 10.1182/blood-2005-02-0516. Epub 2005 May 26. PMID: 15920010; PMCID: PMC1895228.
29. Macchiarini F, Manz M G, Palucka A K, Shultz L D. Humanized mice: are we there yet? J Exp Med. 2005 Nov. 21; 202(10):1307-11. doi: 10.1084/jem.20051547. PMID: 16301740; PMCID: PMC2212979.
30. Shultz L D, Banuelos S, Lyons B, Samuels R, Burzenski L, Gott B, Lang P, Leif J, Appel M, Rossini A, Greiner D. NOD/LtSz-RaglnullPfpnull mice: a new model system with increased levels of human peripheral leukocyte and hematopoietic stem-cell engraftment. Transplantation. 2003 October; 76(7):1036-42. doi: 10.1097/01.TP.0000083041.44829.2C.
31. Islas-Ohlmayer M, Padgett-Thomas A, Domiati-Saad R, Melkus M W, Cravens P D, Martin Mdel P, Netto G, Garcia J V. Experimental infection of NOD/SCID mice reconstituted with human CD34+ cells with Epstein-Barr virus. J Virol. 2004 December; 78(24):13891-900. doi: 10.1128/JVI.78.24.13891-13900.2004. PMID: 15564497; PMCID: PMC533956.
32. Grusby M J, Auchincloss H Jr, Lee R, Johnson R S, Spencer J P, Zijlstra M, Jaenisch R, Papaioannou V E, Glimcher L H. Mice lacking major histocompatibility complex class I and class II molecules. Proc Natl Acad Sci USA. 1993 May 1; 90(9):3913-7. doi: 10.1073/pnas.90.9.3913. PMID: 8483910; PMCID: PMC46416.
33. Roy C J, Warfield K L, Welcher B C, Gonzales R F, Larsen T, Hanson J, David C S, Krakauer T, Bavari S. Human leukocyte antigen-DQ8 transgenic mice: a model to examine the toxicity of aerosolized staphylococcal enterotoxin B. Infect Immun. 2005 April; 73(4):2452-60. doi: 10.1128/IAL73.4.2452-2460.2005. PMID: 15784591; PMCID: PMC1087414.
34. Belizario J E. Immunodeficient Mouse Models: An Overview. The Open Immunology Journal. 2009; 2:79-85.
35. Fan H, Longacre A, Meng F, Patel V, Hsiao K, Koh J S, Levine J S. Cytokine dysregulation induced by apoptotic cells is a shared characteristic of macrophages from nonobese diabetic and systemic lupus erythematosus-prone mice. J Immunol. 2004 Apr. 15; 172(8):4834-43. doi: 10.4049/jimmuno1.172.8.4834. PMID: 15067061.
36. Shultz L D, Lyons B L, Burzenski L M, Gott B, Chen X, Chaleff S, Kotb M, Gillies S D, King M, Mangada J, Greiner D L, Handgretinger R. Human lymphoid and myeloid cell development in NOD/LtSz-scid IL2R gamma null mice engrafted with mobilized human hemopoietic stem cells. J Immunol. 2005 May 15; 174(10):6477-89. doi: 10.4049/jimmunol.174.10.6477. PMID: 15879151.
37. Makino S, Kunimoto K, Muraoka Y, Mizushima Y, Katagiri K, Tochino Y. Breeding of a non-obese, diabetic strain of mice. Jikken Dobutsu. 1980 January; 29(1):1-13. doi: 10.1538/expanim1978.29.1_1. PMID: 6995140.
38. Blunt T, Finnie N J, Taccioli G E, Smith G C, Demengeot J, Gottlieb T M, Mizuta R, Varghese A J, Alt F W, Jeggo P A, Jackson S P. Defective DNA-dependent protein kinase activity is linked to V(D)J recombination and DNA repair defects associated with the murine scid mutation. Cell. 1995 Mar. 10; 80(5):813-23. doi: 10.1016/0092-8674(95)90360-7. PMID: 7889575.
39. Cao X, Shores E W, Hu-Li J, Anver M R, Kelsall B L, Russell S M, Drago J, Noguchi M, Grinberg A, Bloom E T, et al. Defective lymphoid development in mice lacking expression of the common cytokine receptor gamma chain. Immunity. 1995 March; 2(3):223-38. doi: 10.1016/1074-7613(95)90047-0. PMID: 7697543.
40. Ohbo K, Suda T, Hashiyama M, Mantani A, Ikebe M, Miyakawa K, Moriyama M, Nakamura M, Katsuki M, Takahashi K, Yamamura K, Sugamura K. Modulation of hematopoiesis in mice with a truncated mutant of the interleukin-2 receptor gamma chain. Blood. 1996 Feb. 1; 87(3):956-67. PMID: 8562967.
41. International Publication No. WO 2018/209344
42. Pearson T, Greiner D L, Shultz L D. Creation of “humanized” mice to study human immunity. Curr Protoc Immunol. 2008 May; Chapter 15:Unit 15.21. doi: 10.1002/0471142735.im1521s81. PMID: 18491294; PMCID: PMC3023233.
43. Kim K C, Choi B S, Kim K C, Park K H, Lee H J, Cho Y K, Kim S I, Kim S S, Oh Y K, Kim Y B. A Simple Mouse Model for the Study of Human Immunodeficiency Virus. AIDS Res Hum Retroviruses. 2016 February; 32(2):194-202. doi: 10.1089/AID.2015.0211. Epub 2015 Dec. 17. PMID: 26564392; PMCID: PMC4761813.
44. Yaguchi T, Kobayashi A, Inozume T, Morii K, Nagumo H, Nishio H, Iwata T, Ka Y, Katano I, Ito R, Ito M, Kawakami Y Human PBMC-transferred murine MHC class I/II-deficient NOG mice enable long-term evaluation of human immune responses. Cell Mol Immunol. 2018 November; 15(11):953-962. doi: 10.1038/cmi.2017.106. Epub 2017 Nov. 20. PMID: 29151581; PMCID: PMC6207709.
45. Green and Sambrook, Molecular Cloning, A Laboratory Manual, 2012, Cold Spring Harbor Press.
46. Gibson D G, Young L, Chuang R Y, Venter J C, Hutchison C A 3rd, Smith H O. Enzymatic assembly of DNA molecules up to several hundred kilobases. Nat Methods. 2009 May; 6(5):343-5. doi: 10.1038/nmeth.1318. Epub 2009 Apr. 12. PMID: 19363495.
47. Gibson D G, Smith H O, Hutchison C A 3rd, Venter J C, Merryman C. Chemical synthesis of the mouse mitochondrial genome. Nat Methods. 2010 November; 7(11):901-3. doi: 10.1038/nmeth.1515. Epub 2010 Oct. 10. PMID: 20935651.
48. Wang W, Kutny P M, Byers S L, Longstaff C J, DaCosta M J, Pang C, Zhang Y, Taft R A, Buaas F W, Wang H. Delivery of Cas9 Protein into Mouse Zygotes through a Series of Electroporation Dramatically Increases the Efficiency of Model Creation. J Genet Genomics. 2016 May 20; 43(5):319-27. doi: 10.1016/j.jgg.2016.02.004. Epub 2016 Mar. 8. PMID: 27210041; PMCID: PMC4892940.
49. International Publication No. WO 2016/054032
50. International Publication No. WO 2017/124086
51. Carroll D. Genome engineering with zinc-finger nucleases. Genetics. 2011 August; 188(4):773-82. doi: 10.1534/genetics.111.131433. PMID: 21828278; PMCID: PMC3176093.
52. Joung J K, Sander J D. TALENs: a widely applicable technology for targeted genome editing. Nat Rev Mol Cell Biol. 2013 January; 14(1):49-55. doi: 10.1038/nrm3486. Epub 2012 Nov. 21. PMID: 23169466; PMCID: PMC3547402.
53. Gaj T, Gersbach C A, Barbas C F 3rd. ZFN, TALEN, and CRISPR/Cas-based methods for genome engineering. Trends Biotechnol. 2013 Ju1; 31(7):397-405. doi: 10.1016/j.tibtech.2013.04.004. Epub 2013 May 9. PMID: 23664777; PMCID: PMC3694601.
54. Harms D W, Quadros R M, Seruggia D, Ohtsuka M, Takahashi G, Montoliu L, Gurumurthy C B. Mouse Genome Editing Using the CRISPR/Cas System. Curr Protoc Hum Genet. 2014 Oct. 1; 83:15.7.1-27. doi: 10.1002/0471142905.hg1507s83. PMID: 25271839; PMCID: PMC4519007.
55. Inui M, Miyado M, Igarashi M, Tamano M, Kubo A, Yamashita S, Asahara H, Fukami M, Takada S. Rapid generation of mouse models with defined point mutations by the CRISPR/Cas9 system. Sci Rep. 2014 Jun. 23; 4:5396. doi: 10.1038/srep05396. PMID: 24953798; PMCID: PMC4066261.
56. Jinek M, Chylinski K, Fonfara I, Hauer M, Doudna J A, Charpentier E. A programmable dual-RNA-guided DNA endonuclease in adaptive bacterial immunity. Science. 2012 Aug. 17; 337(6096):816-21. doi: 10.1126/science.1225829. Epub 2012 Jun. 28. PMID: 22745249; PMCID: PMC6286148.
57. Deltcheva E, Chylinski K, Sharma C M, Gonzales K, Chao Y, Pirzada Z A, Eckert M R, Vogel J, Charpentier E. CRISPR RNA maturation by trans-encoded small RNA and host factor RNase III. Nature. 2011 Mar. 31; 471(7340):602-7. doi: 10.1038/nature09886. PMID: 21455174; PMCID: PMC3070239.
58. Sukoff Rizzo S J, Anderson L C, Green T L, McGarr T, Wells G, Winter S S.

Assessing Healthspan and Lifespan Measures in Aging Mice: Optimization of Testing Protocols, Replicability, and Rater Reliability. Curr Protoc Mouse Biol. 2018; 8(2):e45. Epub 2018/06/21. doi: 10.1002/cpmo.45. PubMed PMID: 29924918.
59. Jackson H M, Soto I, Graham L C, Carter G W, Howell G R. Clustering of transcriptional profiles identifies changes to insulin signaling as an early event in a mouse model of Alzheimer's disease. BMC Genomics. 2013; 14:831. doi: 10.1186/1471-2164-14-831. PubMed PMID: 24274089; PubMed Central PMCID: PMCPMC3907022.
60. Onos K D, Uyar A, Keezer K J, Jackson H M, Preuss C, Acklin C J, et al. Enhancing face validity of mouse models of Alzheimer's disease with natural genetic variation. PLoS Genet. 2019; 15(5):e1008155. Epub 2019/06/01. doi: 10.1371/journal.pgen.1008155. PubMed PMID: 31150388; PubMed Central PMCID: PMCPMC6576791.

1. An immunocompromised mouse comprising in its genome a loss-of-function mutation in a murine Prkdc gene, a loss-of-function mutation in a murine Il2rg gene, and a nucleic acid encoding a human or humanized amyloid precursor protein (APP).

2. The immunocompromised mouse of claim 1, wherein the immunocompromised mouse has a non-obese diabetic (NOD) genetic background.

3. The immunocompromised mouse of claim 1, wherein the loss-of-function mutation in a murine Prkdc gene is a null mutation in a murine Prkdc gene and wherein the loss-of-function mutation in a murine Il2rg gene is a null mutation in a murine Il2rg gene.

4. The immunocompromised mouse of claim 3, wherein the null mutation in a murine Prkdc gene is a Prkdc^{scid} mutation and wherein the null mutation in a murine Il2rg gene is a Il2rg^{tm1Wjl} mutation or a Il2rg^{em26Cd22} mutation.

5.-6. (canceled)

7. The immunocompromised mouse of claim 4, wherein the immunocompromised mouse has a NOD.Cg-Prkdc^{scid} Il2rg^{tm1Wjl}/SzJ genetic background.

8. (canceled)

9. The immunocompromised mouse of claim 4, wherein the immunocompromised mouse has a NOD-Prkdc^{em26Cd52}Il2rg^{em26Cd22}/NjuCrl genetic background.

10. The immunocompromised mouse of claim 1, comprising a nucleic acid encoding a humanized APP.

11. The immunocompromised mouse of claim 10, wherein the nucleic acid encoding a humanized APP is a chimeric nucleic acid comprising mouse and human coding sequences.

12. The immunocompromised mouse of claim 11, wherein the chimeric nucleic acid comprises a human coding sequence in the A-beta domain of a mouse APP coding sequence.

13. The immunocompromised mouse of claim 11, wherein the chimeric nucleic acid encodes human mutations K595N and M596L, relative to a human APP comprising the amino acid sequence of SEQ ID NO: 1.

14. The immunocompromised mouse of claim 13, wherein the immunocompromised mouse comprises in its genome an APPswe transgene.

15. The immunocompromised mouse of claim 1 further comprising in its genome a nucleic acid encoding a mutated human presenilin 1 protein (PSEN1).

16. The immunocompromised mouse of claim 15, wherein the nucleic encoding a mutated PSEN1 comprises a human PSEN coding sequence that comprises a deletion in exon 9.

17. The immunocompromised mouse of claim 16, wherein the immunocompromised mouse comprises in its genome a PSENde9 transgene.

18. The immunocompromised mouse of claim 17, wherein the immunocompromised mouse comprises in its genome Tg(APPswe,PSENlde9)85Dbo transgene insertion.

19. (canceled)

20. The immunocompromised mouse of claim 1, wherein the immunocompromised mouse has at least one characteristic of early-onset Alzheimer's disease selected from a cognitive deficit, increased hippocampal plaque deposits, and increased neuroinflammation in the brain, relative to a control.

21. (canceled)

22. The immunocompromised mouse of claim 1, wherein the immunocompromised mouse does not have a measurable tumor burden.

23. The immunocompromised mouse of claim 1, wherein the immunocompromised mouse is at least a year old.

24.-28. (canceled)

29. A method, comprising introducing into non-obese diabetic (NOD) mouse a null mutation in a murine Prkdc gene, a null mutation in a murine Il2rg gene, a nucleic acid encoding a human or humanized amyloid precursor protein (APP), and a nucleic encoding a mutated human presenilin 1 protein (PSEN1).

30. (canceled)

31. A method, comprising breeding (a) a non-obese diabetic (NOD) an NOD mouse comprising (i) a loss-of-function mutation in the murine Prkdc gene and (ii) a loss-of-function mutation in a murine Il2rg gene to (b) an NOD mouse comprising (i) a nucleic acid encoding a human or humanized amyloid precursor protein and (ii) a nucleic acid encoding a mutated human presenilin 1 protein, to produce an immunocompromised progeny mouse having characteristics of early-onset Alzheimer's disease.

32.-34. (canceled)
